(19) United States
(12) Patent Application Publication
(10) Pub. No.: US 2017/0071618 A1
(43) Pub. Date: Mar. 16, 2017

(54) Steerable Laparoscopic Instrument

(71) Applicant: CoreBio Technologies Co., Ltd., Taoyuan City (TW)

(72) Inventors: Wen-Ming Hsu, New Taipei City (TW); Chih-Hung Li, Taoyuan City (TW)

(21) Appl. No.: 14/926,808

(22) Filed: Oct. 29, 2015

(30) Foreign Application Priority Data
Sep. 11, 2015 (TW) 104130211

(51) Int. Cl. A61B 17/29

(52) U.S. Cl. CPC A61B 17/29; A61B 2017/00867

(57) ABSTRACT

A steerable laparoscopic instrument is provided. The steerable laparoscopic instrument comprises a transmission part, a first disc, a second disc, a bending part, a third disc, a working part, and a handle part. The steerable laparoscopic instrument provides surgical operation with at least two directional controls, is easily disassembled and reassembled, can be operated by a single hand, and is easily to be cleaned and sterilized.

### FIELD OF THE INVENTION

The present invention relates to a laparoscopic instrument, and more particularly to a steerable laparoscopic instrument.

### BACKGROUND OF THE INVENTION

As shown in FIG. 1, a conventional laparoscopic instrument typically has only a single directional control, i.e., the 360 degree rotational control of tube 1 allowing the working part 2 to work at various angles. The working part 2 may be scissors, pliers, or the like. However, to adjust the working part 2 of such instrument at a proper orientation during minimally invasive surgery would be difficult due to limited operation space.

Therefore, it is urgently desired in this field to develop an improved laparoscopic instrument which provides multiple directional controls for the surgeon to have maximal degree of freedom in performing minimally invasive surgery.

### SUMMARY OF THE INVENTION

Accordingly, the present invention provides a steerable laparoscopic instrument, which has at least two directional controls. The steerable laparoscopic instrument comprises a transmission part, a first disc, a second disc, a bending part, a third disc, a working part, and a handle part.

The transmission part has a first end and a second end, and comprises an inner tube and an outer tube, both of which extend along a first axis.

The first disc is disposed at the first end of the transmission part. The first disc is coupled with the outer tube and is used to control the rotation of the outer tube on the first axis.

The second disc is also disposed at the first end of the transmission part. The second disc is coupled with the inner tube and is used to control the movement of the inner tube along the first axis.

The bending part is pivotally connected to the second end of the transmission part. Further, the bending part has a sliding linkage pivotally connected thereto. The sliding linkage is coupled with the inner tube.

The third disc is disposed at the first end of the transmission part. The third disc comprises a center transmission rod coupled therewith. The center transmission rod extends along the first axis.

The working part is connected to the bending part. The working part extends along a second axis and has a connecting member, a third end, a fourth end and a plier-like member. The connecting member is disposed at the third end, and the plier-like member is disposed at the fourth end. Further, the working part is coupled with the third disc through the center transmission rod.

The handle part is disposed at a side of the first end of the transmission part. The handle part comprises a control member and a handle. The control member is coupled with the center transmission rod and is used to control the movement of the center transmission rod along the first axis.

According to the present invention, it is preferable that at least a section of the center transmission rod is of superelastic material, wherein the section is a section of the center transmission rod that has a position corresponding to the bending part.

In one embodiment of the present invention, the superelastic material is a shape memory alloy.

According to the present invention, the movement of the sliding linkage changes the angle between the first and second axes by moving back and forth along the first axis.

In one embodiment of the present invention, the connecting member connects to the bending part through a buckle.

In certain embodiments of the present invention, the handle part further comprises a locking member. The locking member is disposed at a side of the handle part and is used to lock the control member to the handle in tooth-type, cam-type or spring-type.

In certain embodiments of the present invention, the opening and closing of the plier-like member is controlled by a central screw driven by an inner torsion spring or an outer torsion spring.

According to one embodiment of the present invention, the steerable laparoscopic instrument further comprises a housing for accommodating the first, second and third discs.

### BRIEF DESCRIPTION OF THE DRAWINGS

The foregoing summary, as well as the following detailed description of the invention, will be better understood when read in conjunction with the appended drawing. In the drawings:

FIG. 1 is a schematic view of a conventional laparoscopic instrument.

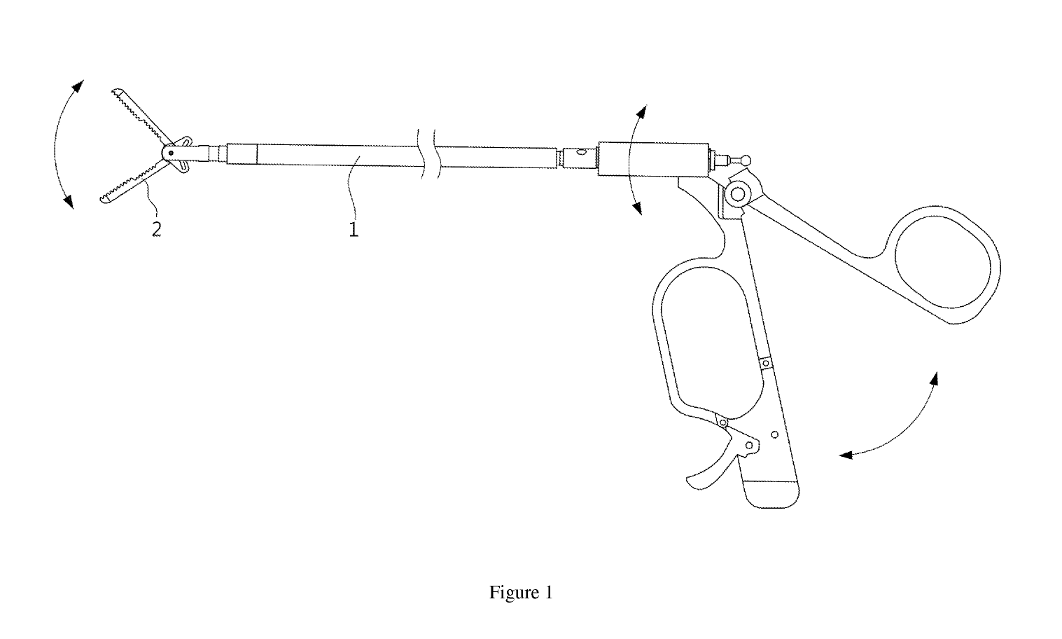

FIG. 2 is a perspective view of a steerable laparoscopic instrument of the present invention.

FIG. 3 shows the relationship between the working part and the bending part of a steerable laparoscopic instrument of the present invention.

FIG. 4 illustrates the locking member of the handle part of a steerable laparoscopic instrument of the present invention.

### DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

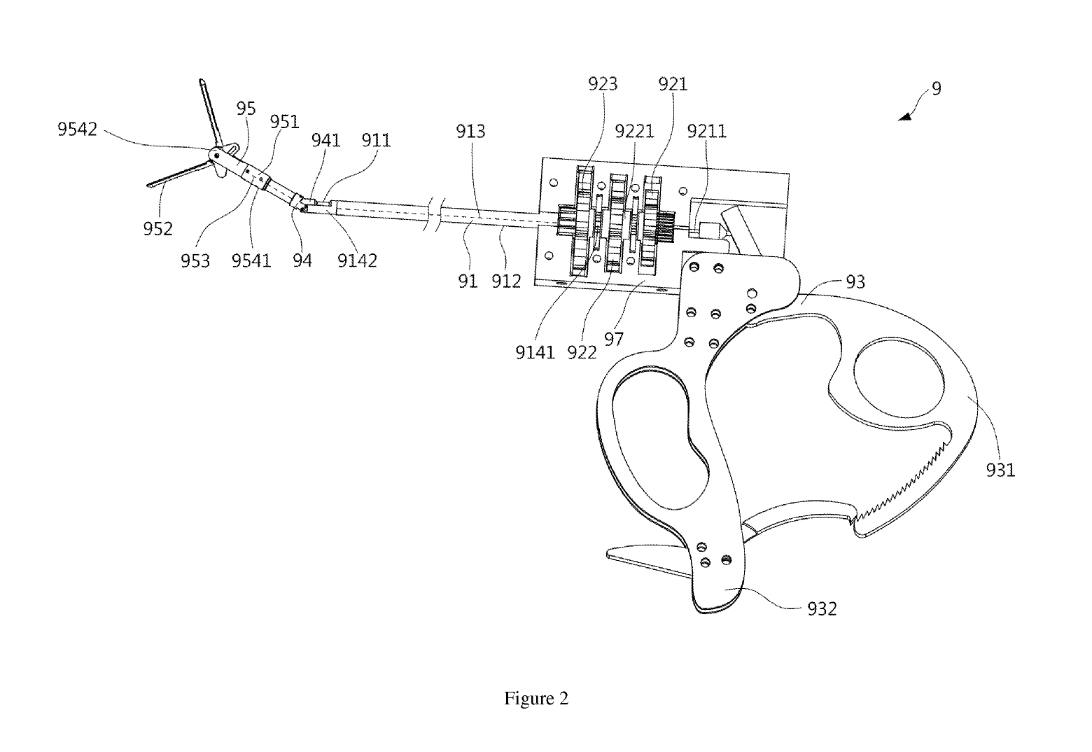

Referring to FIG. 2, shown is an embodiment of the steerable laparoscopic instrument according to the present invention. A steerable laparoscopic instrument 9 comprises a transmission part 91, a first disc 923, a second disc 922, a bending part 94, a third disc 921, a working part 95, a handle part 93, and a housing 97.

The transmission part 91 extends along a first axis 913, and includes an outer tube 912 and an inner tube 911 movably disposed inside the outer tube 912. Both of the inner and outer tubes 911 and 912 extend along the first axis 913. The transmission part 91 has a first end 9141 and a second end 9142.

The first disc 923 is disposed at the first end 9141 of the transmission part 91. The first disc 923 is coupled with the outer tube 912 and is used to control (or manipulate) the rotation of the outer tube 912 on the first axis 913. In other words, the first disc 923 is coupled with the outer tube 912 in a manner such that the rotation of the first disc 923 on the first axis 913 drives the outer tube 912 to rotate on the first axis 913 correspondingly.

The second disc 922 is also disposed at the first end 9141 of the transmission part 91. As shown in FIG. 2, the second disc 922 may be disposed adjacent the first disc 923. In addition, the second disc 922 is coupled with the inner tube 911 in a manner such that the rotation of the second disc 922 on the first axis 913 drives the inner tube 911 to move along the first axis 913 correspondingly. For example, when the second disc 922 is rotated clockwise on the first axis 913, the inner tube 911 moves correspondingly toward the direction of the second end 9142 of the transmission part 91 (to the left in FIG. 2) along the first axis 913; and when the second disc 922 is rotated counterclockwise on the first axis 913, the inner tube 911 moves correspondingly toward the direction of the first end 9141 of the transmission part 91 (to the right in FIG. 2) along the first axis 913. In the present embodiment, the second disc 922 is coupled with the inner tube 911 through a screw nut 9221 which is co-axial with the first axis 913.

In addition, the bending part 94 is pivotally connected to the second end 9142 of the transmission part 91. Specifically, the bending part 94 is pivotally connected to the outer tube 912 of the transmission part 91. A sliding linkage 941 is pivotally connected to the bending part 94. The sliding linkage 941 is coupled with the inner tube 911, such that the movement of the inner tube 911 along the first axis 913 drives the sliding linkage to move along the first axis 913 correspondingly. Accordingly, when rotating the second disc 922 and the screw nut 9221, the movement of the sliding linkage causes the rotation of the bending part 94 with respect to the pivot point where the bending part 94 is pivoted to the second end 9142 of the transmission part 91.

The third disc 921 is disposed at the first end 9141 of the transmission part 91. A center transmission rod 9211 extends along the first axis 913 is coupled with the third disc 921. Preferably, the center transmission rod 9211 is coupled with the third disc 921 in a manner such that the rotation of the third disc 921 on the first axis 913 drives the center transmission rod 9211 to rotate correspondingly on the first axis 913.

The working part 95 is connected to the bending part 94. The working part 95 extends along a second axis 953 and has a connecting member 951, a third end 9541, a fourth end 9542 and a plier-like member 952. The connecting member 951 is disposed at the third end 9541 and the plier-like member 952 is disposed at the fourth end 9542. The working part 95 is coupled with the third disc 921 through the center transmission rod 9211 in a manner such that the rotation of the third disc 921 on the first axis 913 drives the rotation of the working part 95 on the first axis 913 correspondingly. Put differently, the center transmission rod 9211 run through the interior of the transmission part 91, the bending part 94 and the working part 95, and one end of the center transmission rod 9211 is connected to the working part 95 while the other end of the center transmission rod 9211 is connected to the third disc 921.

Further, the handle part 93 is disposed at a side of the first end 9141 of the transmission part 91. The handle part 93 includes a control member 931 and a handle 932. The control member 931 is coupled with the center transmission rod 9211 and is used to control the movement of the center transmission rod 9211 along the first axis 913. The movement of the center transmission rod 9211 along the first axis 913 is for the control of the opening and closing of the plier-like member 952 of the working part 95.

The steerable laparoscopic instrument 9 may further comprise a housing 97 for accommodating the first disc 923, the second disc 922 and the third disc 921. The housing 97 may be connected to the handle 932.

Accordingly, the outer tube 923 (along with the working part 95) is up to 360 degree rotatable on the first axis 913 by rotating the first disc 923; the transmission part 94 (along with the working part 95) is steerable with respect to the first axis 913 by rotating the second disc 923 (the angle between the first and second axes 913 and 953 is changed accordingly); and the working part 95 (along with the plier-like member 952) is up to 360 degree rotatable on the second axis 953, independently from the rotation of the outer tube 923, by rotating the third disc 921.

With reference to FIG. 2 again, in accordance with one preferred embodiment of the present invention, a section of the center transmission rod 9211 having a position corresponding to the bending part 94 is of superelastic (or termed as “pseudoelastic”) material. Alternatively, the whole center transmission rod 9211 may be made of a superelastic material. The superelastic material includes but is not limited to a shape memory alloy, for example, a nickel-titanium alloy. In one embodiment of the present invention, the nickel-titanium alloy used has the following properties: (I) Mechanical properties: (a) tensile strength: 830˜1170 MPa, (b) yield strength: 200 MPa, (c) Young's modulus: 70 GPa, (d) Poisson's ratio: 0.33, and (e) hardness: 80 RB; and (II) Shape memory properties: (a) transformation temperature: −20˜+150° C.; (b) shape memory strength: 550 MPa; and (c) deformation amount: 6%.

Referring to FIG. 2, in accordance with one embodiment of the present invention, the movement of the sliding linkage 941 moves the working part 95 in link-type and changes the angle formed between the first and second axes 913 and 953, wherein said angle may be greater than 90 degrees.

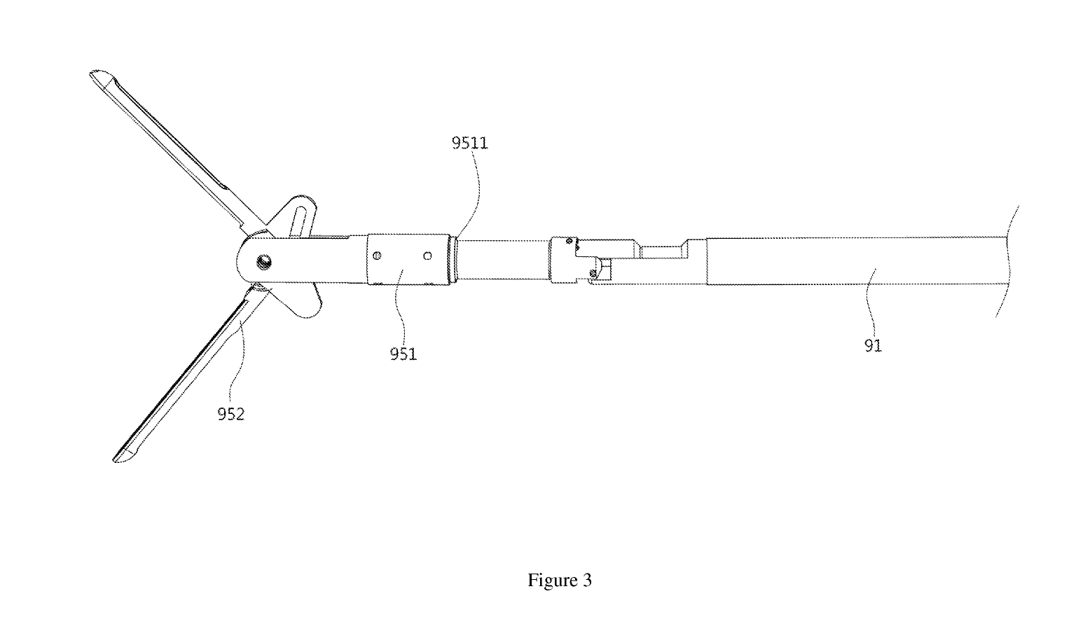

With reference to FIG. 3, in accordance with one embodiment of the present invention, the connecting member 951 removably connects to the bending part 94 through a buckle 9511. The plier-like member 952 may be pliers, scissors, claws pliers or a needle holder, but is not limited thereto.

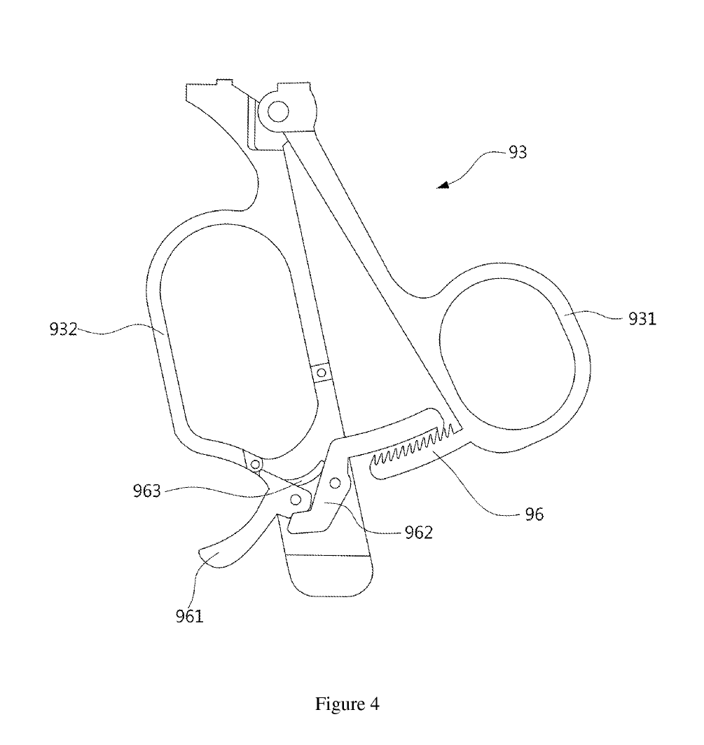

Referring to FIG. 4, the handle part 93 further comprises a locking member 96. The locking member 96 is disposed at a side of the handle part 93 and is used to lock the control member 931 to the handle 932 in tooth-type, cam-type or spring-type. In one preferred embodiment, the locking member 96 comprises a switch 961, a pawl 962, and a spring sheet 963. In operation, when the switch 961 is not pressed, the control member 931 can only be moved in one direction and the handle part 93 is in a locked state; for a semi-unlocked state, the pawl 962 is moved away from the locking member 96 when the switch 961 is pressed, and the control member 931 can be freely moved in both directions to control the opening and closing of the plier-like member 952 with the switch 961 being continuously pressed; and when the switch 961 is pushed upward, the pawl 962 would not bounce back by spring sheet 963 since the locking member 96 is in an irreversible position, and thus the handle part 93 is in an unlocked state.

In one embodiment of the present invention, the opening and closing of the plier-like member 952 is controlled by a central screw driven by an inner torsion spring or an outer torsion spring.

In the development of the present invention, considering the relevance between manipulation and transmission under displacement controlled design, the durability under elastic deformation, wear resistant shape design, assembly processing costs and mechanism durability, the difficulties on the small mechanism space, space limitation and durability of the transmission transition and whether a complicated mechanism can be easily disassembled and reassembled were overcome, and the following advantages were achieved: small limitation on the steering angle, multiple rotation/steering functions, convenience on disassembling, cleaning and replaceability, and high durability.

It is believed that a person of ordinary knowledge in the art where the present invention belongs can utilize the present invention to its broadest scope based on the descriptions herein with no need of further illustration. Therefore, the descriptions and claims as provided should be understood as of demonstrative purpose instead of limitative in any way to the scope of the present invention.

I/we claim:

1. A steerable laparoscopic instrument, comprising:
a transmission part extending along a first axis, including an inner tube and an outer tube extending along said first axis, the transmission part having a first end and a second end;
a first disc disposed at the first end of the transmission part, coupled with the outer tube and used to control the rotation of the outer tube on the first axis;
a second disc disposed at the first end of the transmission part, coupled with the inner tube and used to control the movement of the inner tube along the first axis;
a bending part pivotally connected to the second end of the transmission part, having a sliding linkage pivotally connected thereto, the sliding linkage being coupled with the inner tube;
a third disc disposed at the first end of the transmission part, including a center transmission rod extending along the first axis coupled therewith;
a working part connected to the bending part, which extends along a second axis and has a connecting member, a third end, a fourth end and a plier-like member, the connecting member being disposed at the third end and the plier-like member being disposed at the fourth end, the working part being coupled with the third disc through the center transmission rod; and
a handle part disposed at a side of the first end of the transmission part, including a control member and a handle, the control member being coupled with the center transmission rod and used to control the movement of the center transmission rod along the first axis.

2. The steerable laparoscopic instrument of claim 1, wherein a section of the center transmission rod having a position corresponding to the bending part is of superelastic material.

3. The steerable laparoscopic instrument of claim 2, wherein the superelastic material is a shape memory alloy.

4. The steerable laparoscopic instrument of claim 1, wherein the movement of the center transmission rod along the first axis is used to control the opening and closing of the plier-like member of the working part

5. The steerable laparoscopic instrument of claim 1, wherein the movement of the sliding linkage moves the working part in link-type and changes the angle formed between the first and second axes.

6. The steerable laparoscopic instrument of claim 1, wherein the connecting member connects to the bending part through a buckle.

7. The steerable laparoscopic instrument of claim 1, wherein the handle part further includes a locking member disposed at a side of the handle part, the locking member being used to lock the control member to the handle in tooth-type, cam-type or spring-type.

8. The steerable laparoscopic instrument of claim 1, wherein the opening and closing of the plier-like member is controlled by a central screw driven by an inner torsion spring or an outer torsion spring.

9. The steerable laparoscopic instrument of claim 1, further comprising a housing for accommodating the first, second and third discs.
